(19) United States
(12) Patent Application Publication
(10) Pub. No.: US 2014/0287008 A1
(43) Pub. Date: Sep. 25, 2014

(54) HYBRID POLYUREA FRAGRANCE ENCAPSULATE FORMULATION AND METHOD FOR USING THE SAME

(71) Applicant: International Flavors & Fragrances Inc., New York, NY (US)

(72) Inventors: Yabin Lei, Holmdel, NJ (US); Michael V. Imperiale, Newark, NJ (US); Lewis M. Popplewell, Morganville, NJ (US); Franklin Pringgosusanto, Laurence Harbor, NJ (US); Ralph Gencarelli, Fairfield, NJ (US)

(21) Appl. No.: 14/299,733

(22) Filed: Jun. 9, 2014

Related U.S. Application Data
(63) Continuation-in-part of application No. 14/057,127, filed on Oct. 18, 2013.
Application No. 14/057,127 is a continuation-in-part of application No. 13/422,090, filed on Mar. 16, 2012.
Application No. 13/422,090 is a continuation-in-part of application No. 12/793,911, filed on Jun. 4, 2010.
Application No. 12/793,911 is a continuation-in-part of application No. 12/328,340, filed on Dec. 4, 2008, now abandoned.
(60) Provisional application No. 61/453,977, filed on Mar. 18, 2011.

(51) Int. Cl. A61K 8/11; A61Q 5/02; A61Q 11/00; A61Q 19/10; A61Q 13/00; C11D 3/50; A61Q 15/00; A61Q 5/12

(52) U.S. Cl. CPC A61K 8/11; A61Q 15/00; A61Q 5/02; A61Q 5/12; A61Q 19/10; A61Q 13/00; C11D 3/505; A61Q 11/00; A61K 2008/115
USPC 424/401; 424/65; 510/119; 424/70.13; 510/130; 512/4; 510/349; 424/49; 264/4.3

(57) ABSTRACT

A hybrid polyurea encapsulate formulation obtained by mixing a starch/fragrance emulsion with a polyurea capsule suspension is provided as is a method of using the formulation in a personal care product, a beauty care product, a fabric care product, a home care product, a personal hygiene product, an oral care product and a method for releasing an encapsulated fragrance by moisture, shear, or a combination thereof.

### CROSS-REFERENCES

This application is a continuation-in-part of U.S. application Ser. No. 14/057,127, filed Oct. 18, 2013, which is a continuation-in-part of U.S. application Ser. No. 13/422,090, filed Mar. 16, 2012, which claims benefit of priority from U.S. Provisional Application Ser. No. 61/453,977, filed Mar. 18, 2011; and is a continuation-in-part of U.S. application Ser. No. 12/793,911, filed Jun. 4, 2010, which is continuation-in-part of U.S. application Ser. No. 12/328,340, now abandoned, filed Dec. 4, 2008, the contents of which are incorporated herein by reference in their entireties.

### BACKGROUND

Spray-drying is a well known technique for the encapsulation of flavors and fragrances. Spray-dried products are commonly prepared from an emulsion that is sprayed into a drying chamber. A number of emulsion parameters influence the quality of the spray-dried capsules. For example, to achieve a spray-dried product of relatively small droplet size, the emulsion preferably remains stable during the duration of the spray-drying process, which can vary from a few minutes to several hours. The stability of the droplet size in the emulsion is even more important and difficult to achieve when high amounts of flavors or fragrances are intended to be encapsulated.

In certain instances, biopolymers with surface active properties, e.g., Gum arabic, starches, cellulose, gelatin, alginates, and proteins including albumin or beta-globulin, are used as emulsifiers. For example, US Patent Application 2009/0253612 describes a spray-dry encapsulation process for flavor or fragrance comprising drying an aqueous emulsion containing the oil to be encapsulated, modified starch and phosphate salts. Furthermore, an antiperspirant/deodorant containing microcapsules is disclosed in U.S. Pat. No. 5,176,903, where a fragrance oil and ester are encapsulated by a food starch and polysaccharide composition.

### SUMMARY OF THE INVENTION

One aspect of this invention relates to a hybrid encapsulate formulation obtained by a method including the following steps: (i) preparing an aqueous starch solution; (ii) preparing an oil phase containing an active material; (iii) emulsifying the oil phase with an aqueous starch solution to obtain an emulsion; mixing the emulsion with a polyurea capsule suspension; and (iv) spray-drying the mixture to obtain a hybrid encapsulate formulation. In some embodiments, the aqueous starch solution further includes maltose, sucrose, maltodextrin, or a combination thereof. In other embodiments, the oil phase optionally includes monoglycerides, lecithin, or a combination thereof. In further embodiments, a salt is optionally added to the polyurea capsule suspension and subsequently washed with water prior to being mixed with the emulsion. In still further embodiments, the active material is a fragrance oil. In yet other embodiments, the polyurea capsule suspension encapsulates an active material, e.g., a fragrance oil. In some embodiments, the polyurea capsule suspension includes a nonionic polymer (e.g., polyvinylpyrrolidone, polyvinyl alcohol, polyethylene glycol, polyethylene oxide, polyethylene oxide-polypropylene oxide, polyethylene oxide-polypropylene oxide-polyethylene oxide, and a combination thereof), cationic polymer (e.g., Polyquaterium-6, Polyquaternium-11, Polyquaternium-47, and a combination thereof), anionic polymer (e.g., a polystyrene sulfonic acid, polyacrylic acid, hyaluronic acid, sodium alginate, sodium carboxymethylcellulose, and a combination thereof), anionic surfactant (e.g., sodium laureth sulfate, complex ester of phosphoric acid and ethoxylated cosmetic grade oleyl alcohol, and a combination thereof), or a combination thereof. In other embodiments, the ratio of starch/polyuria is 10/90 to 90/10 on a dry weight basis).

Another aspect of this invention relates to a personal care product (e.g., an aerosol antiperspirant, stick antiperspirant, roll-on antiperspirant, emulsion spray antiperspirant, clear emulsion stick antiperspirant, soft solid antiperspirant, emulsion roll-on antiperspirant, clear emulsion stick antiperspirant, opaque emulsion stick antiperspirant, clear gel antiperspirant, clear stick deodorant or spray deodorant, shampoo, hair conditioner, hair rinse, hair refresher, body wash, and soap), a beauty care product (e.g., fine fragrance and Eau De Toilette), a fabric care product (e.g., a rinse conditioner, liquid detergent, and powder detergent), a home care product (e.g., an all-purpose cleaner and fabric refresher), a personal hygiene product (e.g., hand sanitizer), or an oral care product (e.g., tooth powder), each of which contains the hybrid encapsulate formulation described above. Also within the scope of this invention are a method for preparing the hybrid encapsulate formulation and a method for releasing an encapsulated fragrance by moisture, shear, or a combination thereof.

### BRIEF DESCRIPTION OF THE DRAWINGS

FIG. 1 shows the performance of a hybrid polyurea capsule formulation composed of 46% ICON fragrance at 0.1% (fragrance equivalent load) in an antiperspirant aerosol base (Test Sample) compared to a Benchmark product.

FIG. 2 shows the performance of a hybrid polyurea capsule formulation composed of TORNADO fragrance at 0.25% NOE in antiperspirant aerosol base (Test Sample) compared to a Benchmark product.

FIG. 3 shows the performance of a hybrid polyurea capsule formulation composed of No LIMIT fragrance at 0.25% NOE in antiperspirant aerosol base (Test Sample) compared to a Benchmark product.

### DETAILED DESCRIPTION OF THE INVENTION

It is often desired to control the release of a perfume or flavor out of a consumer product, in particular when the perfumed or flavored consumer product is intended to produce consumer conceivable benefits at a certain “magic moment” under a wide variety of user environments. This invention relates to hybrid encapsulate formulations that can provide variable and multistage release of encapsulated materials. The release of encapsulated active materials, such as fragrances, can be triggered either by moisture or shear to provide multi-stage release profiles. The hybrid formulations are suitable for a wide range of personal applications including, but not limited to, antiperspirant and deodorant products.

Accordingly, within the scope of this invention is a hybrid encapsulate formulation obtained by (i) preparing an aqueous starch solution; (ii) preparing an oil phase containing an active material; (iii) emulsifying the oil phase with the aqueous starch solution to obtain a fragrance emulsion; (iv) mixing the fragrance emulsion with a polyurea, core-shell capsule suspension; and (v) spray-drying the mixture. In accordance with the present invention, an aqueous solution refers to a solution in which the solvent is water. As is known in the art, the term “starch” refers to a carbohydrate composed of a large number of glucose units joined by glycosidic bonds. The starch can be obtained from grains, grasses, tubers, and roots by wet grinding, washing, sieving, or drying. Starches are predominantly obtained from corn, wheat and potato, and to a lesser extent, sources such as rice, sweet potato, sago and mung bean. The starch can be unmodified or chemically modified to allow the starch to function under conditions frequently encountered during processing or storage, such as high heat, high shear, low pH, oxidation, freeze/thaw, and cooling. Such modifications include, but are not limited to, acid treatment, alkaline treatment, bleaching, oxidation, enzyme treatment, acetylation, phosphorylation, and a combination thereof. Typical modified starches include cationic starches, hydroxyethyl starch and carboxymethylated starches. In some embodiments, the starch is a modified starch. Exemplary modified starches include, but are not limited to, CAPSUL, CAPSUL FP, HI-CAP IMF, HI-CAP 100, and the combination thereof. In other embodiments, the aqueous starch solution optionally includes maltose, sucrose, maltodextrin, or a combination thereof. In still other embodiments, the aqueous starch solution optionally includes a cellulose ether, e.g., Methocel.

The oil phase of this invention includes oil soluble ingredients. The oil phase can be composed of an active material alone (e.g., a fragrance oil) or include one or more other components such as a surfactant and an emulsifier. In some embodiments, the oil phase includes the active material in combination with a monoglyceride, lecithin, or a combination thereof. In certain embodiments, the active material is a fragrance oil, essential oil, plant extract, or mixture thereof.

In some embodiments, the active material is also encapsulated within a polyurea, core-shell capsule. In certain embodiments, the active material is a fragrance oil, essential oil, plant extract, or mixture thereof. In this respect, the hybrid encapsulate formulation can include a first fragrance (encapsulated in a polyurea capsule) and a second fragrance (present in the oil phase). In some embodiments, the first and second fragrances are the same. In other embodiments, the first and second fragrances are different.

In accordance with the present invention, the ratio of starch/polyurea capsule used in the formulation of this invention is in the range of 10/90 to 90/10 (on a dry weight basis, as provided in the examples below). In certain embodiments, the ratio of starch/polyurea capsule is 10/90, 20/80, 30/70, 40/60, 50/50, 60/40, 70/30, 80/20 or 90/10.

Given the variable and multistage release of encapsulated active materials by moisture or shear triggers, this invention also provides a method for releasing an encapsulated fragrance by moisture, shear, or a combination thereof by (i) encapsulating a first fragrance in a polyurea, core-shell capsule, (ii) mixing the polyurea, core-shell encapsulated fragrance with a fragrance emulsion containing a second fragrance and starch to obtain a hybrid fragrance encapsulate formulation, (iii) spray-drying the hybrid fragrance encapsulate formulation, (iv) incorporating the hybrid fragrance encapsulate formulation into a consumer product base to obtain a consumer product, (v) applying the consumer product containing the hybrid fragrance encapsulate to a surface, and (vi) exposing the surface to moisture, shear, or a combination thereof so that the encapsulated fragrance is released. In some embodiments, the encapsulated materials are released in two stages. In one embodiment, the encapsulated material is first released by moisture and then by shear. In a second embodiment, the encapsulated ingredient is first released by shear and then by moisture. The fragrance encapsulate can provide instant release of fragrance by either moisture activation or shear force depending on the environments and application needs. It will also enable the release of encapsulated fragrance ingredients at different time points by shear. Therefore, the invention provide a system that can provide perfumery benefits at different consumer needed “magic moments” by varying release mechanism and release at different application points under a range of application environments.

Encapsulation of active material such as fragrances is known in the art, see for example U.S. Pat. Nos. 2,800,457, 3,870,542, 3,516,941, 3,415,758, 3,041,288, 5,112,688, 6,329,057, and 6,261,483. Polyurea capsules have been used for encapsulation. More specifically, isocyanate-based capsule wall technologies are disclosed in WO 2004/054362; EP 0 148149; EP 0 017 409 BI; U.S. Pat. No. 4,417,916, U.S. Pat. No. 4,124,526, U.S. Pat. No. 5,583,090, U.S. Pat. No. 6,566,306, U.S. Pat. No. 6,730,635, WO 90/08468, WO 92/13450, U.S. Pat. No. 4,681,806, U.S. Pat. No. 4,285,720, U.S. Pat. No. 6,340,653 and U.S. Pat. No. 8,299,011.

Suitable isocyanates include 1,5-naphthylene diisocyanate, 4,4′-diphenylmethane diisocyanate (MDI), hydrogenated MDI (HI2MDI), xylylene diisocyanate (XDI), tetramethylxylol diisocyanate (TMXDI), 4,4′-diphenyldimethylmethane diisocyanate, di- and tetraalkyldiphenylmethane diisocyanate, 4,4′-dibenzyl diisocyanate, 1,3-phenylene diisocyanate, 1,4-phenylene diisocyanate, the isomers of tolylene diisocyanate (TDI), optionally in a mixture, 1-methyl-2,4-diisocyanatocyclohexane, 1,6-diisocyanato-2,2,4-trimethylhexane, 1,6-diisocyanato-2,4,4-trimethylhexane, 1-isocyanatomethyl-3-isocyanato-1,5,5-trimethylcyclohexane, chlorinated and brominated diisocyanates, phosphorus-containing diisocyanates, 4,4′-diisocyanatophenylperfluoroethane, tetramethoxybutane 1,4-diisocyanate, butane 1,4-diisocyanate, hexane 1,6-diisocyanate (HDI), dicyclohexylmethane diisocyanate, cyclohexane 1,4-diisocyanate, ethylene diisocyanate, phthalic acid bisisocyanatoethyl ester, also polyisocyanates with reactive halogen atoms, such as 1-chloromethylphenyl 2,4-diisocyanate, 1-bromomethylphenyl 2,6-diisocyanate, and 3,3-bischloromethyl ether 4,4′-diphenyldiisocyanate. Sulfur-containing polyisocyanates are obtained, for example, by reacting hexamethylene diisocyanate with thiodiglycol or dihydroxydihexyl sulfide. Further suitable diisocyanates are trimethylhexamethylene diisocyanate, 1,4-diisocyanatobutane, 1,2-diisocyanatododecane and dimer fatty acid diisocyanate.

To facilitate wall formation, polyurea capsules can also include cross-linking agents, such as amines or alcohols. Examples of amines include guanidine amines/salts, amphoteric amines, diamines, and a combination thereof.

Water soluble diamines can be used. One class of these amines has a formula of the following:

H₂N(CH₂)ₙNH₂

where n is ≧1. Examples include methylenediamine, ethylenediamine, 1,3-diaminopropane, 1,4-diaminobutane, hexanethylene diamine, hexamethylene diamine, and pentaethylenehexamine. In some embodiments of this invention, n is 6, where the amine is a hexamethylene diamine.

Amines that have more than two (e.g., 3) NH₂ groups can provide a degree of cross linking in the shell wall. Examples include the polyalykylene polyamines of the following formula:

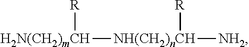

where R is hydrogen or —CH₃, m is 1-5, and n is 1-5. Suitable polyamines include diethylene triamine, triethylene tetraamine, and bis(3-aminopropyl)amine, bis(hexamethylene)triamine.

Another class of amine that can be used in the invention is polyetheramines. They contain primary amino groups attached to the end of a polyether backbone. The polyether backbone is typically based on either propylene oxide (PO), ethylene oxide (EO), or mixed PO and EO. The ether amine can be monoamine, diamine, or triamine. An example is shown below:

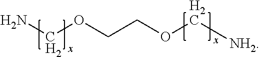

### Exemplary polyetheramines include 2,2′-ethylenedioxy)bis (ethylamine) and 4,7,10-trioxa-1,13-tridecanediamine

Other suitable amines include, but are not limited to, tris(2-aminoethyl)amine, triethylenetetramine, N,N′-bis(3-aminopropyl)-1,3-propanediamine, tetraethylene pent-amine, 1,2-diaminopropane, N,N,N′,N′-tetrakis(2-hydroxy-ethyl)ethylene diamine, N,N,N′,N′-tetrakis(2-hydroxy-propyl)ethylene diamine, branched polyethylenimine, 2,4-diamino-6-hydroxypyrimidine and 2,4,6-triaminopyrimidine.

Amphoteric amines, i.e., amines that can react as an acid as well as a base, are another class of amines of use in this invention. Examples of amphoteric amines include proteins and amino acids such as gelatin, L-lysine, L-arginine, L-lysine monohydrochloride, arginine monohydrochloride and ornithine monohydrochloride.

Guanidine amines and guanidine salts are yet another class of amines of use in this invention. Exemplary guanidine amines and guanidine salts include, but are not limited to, 1,3-diaminoguanidine monohydrochloride, 1,1-dimethylbiguanide hydrochloride, guanidine carbonate and guanidine hydrochloride.

Commercially available amines that can be used include JEFFAMINE EDR-148 (where x=2), JEFFAMINE EDR-176 (where x=3) (from Huntsman). Other polyether amines include the JEFFAMINE ED Series, and JEFFAMINE TRIAMINES.

Alcohols of use as cross-linking agents typically have at least two nucleophilic centers. Exemplary alcohols include, but are not limited to, ethylene glycol, hexylene glycol, pentaerythritol, glucose, sorbitol, and 2-aminoethanol.

As indicated, the polyurea capsules of this invention can be prepared by conventional methods to encapsulate one or more active materials. In some embodiments, the active material is encapsulated by a polymer in the presence of a capsule formation aid, e.g., a surfactant or dispersant. Surfactants or dispersants can be used to make the compositions of this invention more stable. Examples of these surfactants and dispersants include maleic-vinyl copolymers such as the copolymers of vinyl ethers with maleic anhydride or acid, sodium lignosulfonates, maleic anhydride/styrene copolymers, ethylene/maleic anhydride copolymers, and copolymers of propylene oxide, ethylenediamine and ethylene oxide, polyvinylpyrrolidone, polyvinyl alcohols, carboxymethyl cellulose, fatty acid esters of polyoxyethylenated sorbitol and sodium dodecylsulfate.

Commercially available surfactants include, but are not limited to, sulfonated naphthalene-formaldehyde condensates such as MORWET D425 (Akzo Nobel); partially hydrolyzed polyvinyl alcohols such as MOWIOLs, e.g., MOWIOL 3-83 (Air Products); ethylene oxide-propylene oxide block copolymers or poloxamers such as PLURONIC, SYNPERONIC or PLURACARE materials (BASF); sulfonated polystyrenes such as FLEXAN II (Akzo Nobel); and ethylene-maleic anhydride polymers such as ZEMAC (Vertellus Specialties Inc.).

Typically, hydrocolloids or adjuvants are used to improve the colloidal stability of the capsule suspension or slurry against coagulation, sedimentation and creaming. As such, such processing aids can also be used in conjunction with the microcapsules of this invention. As used herein, the term “hydrocolloid” refers to a broad class of water-soluble or water-dispersible polymers having anionic, cationic, zwitterionic, or nonionic character. In particular embodiments, the capsule suspension includes a nonionic polymer, cationic polymer, anionic polymer, anionic surfactant, or a combination thereof. In certain embodiments, the nonionic polymer is a polyvinylpyrrolidone (PVP), polyvinyl alcohol (PVA) polyethylene glycol (PEG), Polyethylene oxide (PEO), or polyethylene oxide-polypropylene oxide (PEO-PPO), polyethylene oxide-polypropylene oxide-polyethylene oxide (PEO-PPO-PEO). In other embodiments, the cationic polymer is Polyquaterium-6 (polydiallyldimethylammonium chloride), Polyquaternium-11 (vinyl pyrrolidone/dimethylaminoethyl methacrylate copolymer) or Polyquaternium-47 (acrylic acid/methacrylamidopropyl trimethyl ammonium chloride/methyl acrylate terpolymer). In yet other embodiments, the anionic polymer is a polystyrene sulfonic acid, polyacrylic acid, hyaluronic acid, sodium alginate, or sodium carboxymethylcellulose (CMC). In still other embodiments, the anionic surfactant is sodium laureth sulfate (SLS) or a complex ester of phosphoric acid and ethoxylated cosmetic grade oleyl alcohol (e.g., CRODAFOS 010A-SS-(RB)).

Other hydrocolloids useful in the present invention include polycarbohydrates, such as starch, modified starch, dextrin, maltodextrin, and cellulose derivatives, and their quaternized forms; natural gums such as alginate esters, carrageenan, xanthanes, agar-agar, pectins, pectic acid, and natural gums such as gum arabic, gum tragacanth and gum karaya, guar gums and quaternized guar gums; gelatin, protein hydrolysates and their quaternized forms; synthetic polymers and copolymers, such as poly(vinyl pyrrolidone-co-vinyl acetate), poly(vinyl alcohol-co-vinyl acetate), poly((met)acrylic acid), poly(maleic acid), poly(alkyl-(meth)acrylate-co-(meth)acrylic acid), poly(acrylic acid-co-maleic acid)copolymer, poly(alkyleneoxide), poly(vinyl-methylether), poly(vinylether-co-maleic anhydride), and the like, as well as poly-(ethyleneimine), poly((meth)-acrylamide), poly(alkyleneoxide-co-dimethylsiloxane), poly-(amino dimethylsiloxane), and their quartenized forms.

The capsule formation aid may also be used in combination with carboxymethyl cellulose and/or a surfactant during processing to facilitate capsule formation. Examples of surfactants that can be used include, but are not limited to, cetyl trimethyl ammonium chloride (CTAC), poloxamers such as PLURONICS (e.g., PLURONIC F127), PLURAFAC (e.g., PLURAFAC F127), or MIRANET-N, saponins such as QNATURALE (National Starch Food Innovation); or a gum Arabic such as Seyal or Senegal. The amount of surfactant present in the capsule slurry can vary depending on the surfactant used. In some embodiments the amount of surfactant is in the range of 0.05 to 0.2 weight percent, in particular when CTAC is employed. In other embodiments, the amount of surfactant is in the range of 1 to 3 weight percent when a saponin or gum arabic is used.

When combined with carboxymethyl cellulose (also referred to as CMC), the lighter color polyvinyl alcohol is preferred. In certain embodiments, the CMC polymer has a molecular weight range between about 90,000 Daltons to 1,500,000 Daltons, more preferably between about 250,000 Daltons to 750,000 Daltons and most preferably between 400,000 Daltons to 750,000 Daltons. The CMC polymer has a degree of substitution between about 0.1 to about 3, more preferably between about 0.65 to about 1.4, and most preferably between about 0.8 to about 1.0.

The CMC polymer is present in the capsule slurry at a level from about 0.1 weight percent to about 2 weight percent and more preferably from about 0.3 weight percent to about 0.7 weight percent.

In some embodiments, CMC-modified microcapsules may provide a perceived fragrance intensity increase of greater than about 15%, and more preferably an increase of greater than about 25% as compared to microcapsules not including CMC.

The diameter of the capsules produced in accordance with this invention can vary from about 10 nanometers to about 1000 microns, preferably from about 50 nanometers to about 100 microns and most preferably from about 2 to about 15 microns. The capsule distribution can be narrow, broad, or multi-modal. Multi-modal distributions may be composed of different types of capsule chemistries.

In some embodiments, the polyurea capsule suspension used in accordance with the present invention is purified. Purification can be achieved by washing the capsule slurry with water, e.g., deionized or double deionized water, until a neutral pH is achieved. For the purposes of the present invention, the polyurea capsule suspension can be washed using any conventional method including the use of a separatory funnel, filter paper, centrifugation and the like. The polyurea capsule suspension can be washed one or more times (e.g., 2-10 times) until a neutral pH, i.e., pH 7±0.5, is achieved. The pH of the purified capsules can be determined using any conventional method including, but not limited to, pH paper, a pH indicator, and a pH meter.

A polyurea capsule suspension of this invention is “purified” in that it is 80-99% (e.g., 90-99%, 95-99%, and 97-99%) homogeneous to polyurea capsules. In accordance with the present invention, purity is achieved by washing the capsules until a neutral pH is achieved, which is indicative of removal of unwanted impurities and/or starting materials, e.g., polyisocyanate, cross-linking agent and the like.

In certain embodiments, the purification of the polyurea capsules includes a step of adding a salt to the polyurea capsule suspension prior to the step of washing the polyurea capsule suspension with water. Exemplary salts include, but are not limited to, sodium chloride, potassium chloride or bi-sulphite salts.

Active Material. Active materials suitable for use in this invention include, without limitation, any combination of fragrance oil, essential oil, plant extract or mixture thereof that is compatible with, and capable of being encapsulated by, a polymer. Individual perfume ingredients that can be included in the capsules of this invention include fragrances containing:

i) hydrocarbons, such as, for example, 3-carene, α-pinene, β-pinene, α-terpinene, γ-terpinene, p-cymene, bisabolene, camphene, caryophyllene, cedrene, farnesene, limonene, longifolene, myrcene, ocimene, valencene, (E,Z)-1,3,5-undecatriene, styrene, and diphenylmethane;

ii) aliphatic alcohols, such as, for example, hexanol, octanol, 3-octanol, 2,6-dimethylheptanol, 2-methyl-2-heptanol, 2-methyl-2-octanol, (E)-2-hexenol, (E)- and (Z)-3-hexenol, 1-octn-3-ol, a mixture of 3,4,5,6,6-pentamethyl-3/4-hepten-2-ol and 3,5,6,6-tetramethyl-4-methyleneheptan-2-ol, (E,Z)-2,6-nonadienol, 3,7-dimethyl-7-methoxy-octan-2-ol, 9-decenol, 10-undecenol, 4-methyl-3-decen-5-ol, aliphatic aldehydes and their acetals such as for example hexanal, heptanal, octanal, nonanal, decanal, undecanal, dodecanal, tridecanal, 2-methyloctanal, 2-methylnonanal, (E)-2-hexenal, (Z)-4-heptenal, 2,6-dimethyl-5-heptenal, 10-undecenal, (E)-4-decenal, 2-dodecenal, 2,6,10-trimethyl-5,9-undecadienal, heptanal-diethylacetal, 1,1-dimethoxy-2,2,5-trimethyl-4-hexene, and citronellyl oxyacetaldehyde;

iii) aliphatic ketones and oximes thereof, such as, for example, 2-heptanone, 2-octanone, 3-octanone, 2-nonanone, 5-methyl-3-heptanone, 5-methyl-3-heptanone oxime, 2,4,4,7-tetramethyl-6-octen-3-one, aliphatic sulfur-containing compounds (e.g., 3-methylthiohexanol, 3-methylthiohexyl acetate, 3-mercaptohexanol, 3-mercaptohexyl acetate, 3-mercaptohexyl butyrate, 3-acetylthiohexyl acetate, and 1-menthene-8-thiol, and aliphatic nitriles (e.g., 2-nonenenitrile, 2-tridecenenitrile, 2,12-tridecenenitrile, 3,7-dimethyl-2,6-octadiencnitrile, and 3,7-dimethyl-6-octenenitrile);

iv) aliphatic carboxylic acids and esters thereof; such as, for example, (E)- and (Z)-3-hexenylformate, ethyl acetoacetate, isoamyl acetate, hexyl acetate, 3,5,5-trimethylhexyl acetate, 3-methyl-2-butenyl acetate, (E)-2-hexenyl acetate, (E)- and (Z)-3-hexenyl acetate, octyl acetate, 3-octyl acetate, 1-octan-3-yl acetate, ethyl butyrate, butyl butyrate, isoamyl butyrate, hexylbutyrate, (E)- and (Z)-3-hexenyl isobutyrate, hexyl crotonate, ethylisovalerate, ethyl-2-methyl pentanoate, ethyl hexanoate, allyl hexanoate, ethyl heptanoate, allyl heptanoate, ethyl octanoate, ethyl-(E,Z)-2,4-decadienoate, methyl-2-octinate, methyl-2-noninate, allyl-2-isoamyl oxyacetate, and methyl-3,7-dimethyl-2,6-octadienoate;

v) acyclic terpene alcohols, such as, for example, citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol 2,6-dimethyl-2,5,7-octatrien-1-ol; as well as formates, acetates, propionates, isobutyrates, butyrates, isovalerates, pentanoates, hexanoates, crotonates, tiglinates and 3-methyl-2-butenoates thereof;

vi) acyclic terpene aldehydes and ketones, such as, for example, geranial, neral, citronellal, 7-hydroxy-3,7-dimethyloctanal, 7-methoxy-3,7-dimethyloctanal, 2,6,10-trimethyl-9-undecenal, α-sinensal, β-sinensal, geranylacetone, as well as the dimethyl- and diethylacetals of geranial, neral and 7-hydroxy-3,7-dimethyloctanal;

vii) cyclic terpene alcohols, such as, for example, menthol, isopulegol, alpha-terpineol, terpinen-4-ol, menthan-8-ol, menthan-1-ol, menthan-7-ol, borneol, isoborneol, linalool oxide, nopol, cedrol, ambrinol, vetiverol, guaiol, and the formates, acetates, propionates, isobutyrates, butyrates, isovalerates, pentanoates, hexanoates, crotonates, tiglinates and 3-methyl-2-butenoates of alpha-terpineol, terpinen-4-ol, methan-8-ol, methan-1-ol, methan-7-ol, borneol, isoborneol, linalool oxide, nopol, cedrol, ambrinol, vetiverol, and guaiol;

viii) cyclic terpene aldehydes and ketones, such as, for example, menthone, isomenthone, 8-mercaptomenthan-3-one, carvone, camphor, fenchone, α-ionone, β-ionone, α-n-methylionone, β-n-methylionone, α-isomethylionone, β-isomethylionone, alpha-irone, α-damascone, β-damascone, β-damascenone, δ-damascone, γ-damascone, 1-(2,4,4-trimethyl-2-cyclohexen-1-yl)-2-buten-1-one, 1,3,4,6,7,8a-hexahydro-1,1,5,5-tetramethyl-2H-2,4a-methanonaphthalen-8(5H—)-one, nootkatone, dihydronootkatone; acetylated cedarwood oil (cedryl methyl ketone);

ix) cyclic alcohols, such as, for example, 4-tert-butylcyclohexanol, 3,3,5-trimethylcyclohexanol, 3-isocamphylcyclohexanol, 2,6,9-trimethyl-Z2,Z5,E9-cyclo-dodecatrien-1-ol, 2-isobutyl-4-methyltetrahydro-2H-pyran-4-ol;

x) cycloaliphatic alcohols, such as, for example, alpha, 3,3-trimethylcyclo-hexylmethanol, 2-methyl-4-(2,2,3-trimethyl-3-cyclopent-1-yl)butanol, 2-methyl-4-(2,2,3-trimethyl-3-cyclopent-1-yl)-2-buten-1-ol, 2-ethyl-4-(2,2,3-trimethyl-3-cyclopent-1-yl)-2-buten-1-ol, 3-methyl-5-(2,2,3-trimethyl-3-cyclopent-1-yl)-pentan-2-ol, 3-methyl-5-(2,2,3-trimethyl-3-cyclopent-1-yl)-4-penten-2-ol, 3,3-dimethyl-5-(2,2,3-trimethyl-3-cyclopent-1-yl)-4-penten-2-ol, 1-(2,2,6-trimethylcyclohexyl)pentan-3-ol, 1-(2,2,6-trimethylcyclohexyl)hexan-3-ol;

xi) cyclic and cycloaliphatic ethers, such as, for example, cineole, cedryl methyl ether, cyclododecyl methyl ether,

xii) (ethoxymethoxy)cyclododecane; alpha-cedrene epoxide, 3a,6,6,9a-tetramethyldodecahydronaphtho[2,1-b]furan, 3a-ethyl-6,6,9a-trimethyldodecahydro-naphtho[2,1-b]furan, 1,5,9-trimethyl-13-oxabicyclo[10.1.0]-trideca-4,8-diene, rose oxide, 2-(2,4-dimethyl-3-cyclohexen-1-yl)-5-methyl-5-(1-methylpropyl)-1,3-dioxan-;

xiii) cyclic ketones, such as, for example, 4-tert-butylcyclohexanone, 2,2,5-trimethyl-5-pentylcyclopentanone, 2-heptylcyclopentanone, 2-pentylcyclopentanone, 2-hydroxy-3-methyl-2-cyclopenten-1-one, 3-methyl-cis-2-penten-1-yl-2-cyclopenten-1-one, 3-methyl-2-pentyl-2-cyclopenten-1-one, 3-methyl-4-cyclopentadecenone, 3-methyl-5-cyclopentadecenone, 3-methylcyclopentadecanone, 4-(l-ethoxyvinyl)-3,3,5,5-tetramethylcyclohexanone, 4-tert-pentylcyclohexanone, 5-cyclohexadecen-1-one, 6,7-dihydro-1,1,2,3,3-pentamethyl-4(5H)-indanone, 5-cyclohexadecen-1-one, 8-cyclohexadecen-1-one, 9-cycloheptadecen-1-one, cyclopentadecanone, cycloaliphatic aldehydes, such as, for example, 2,4-dimethyl-3-cyclohexene carbaldehyde, 2-methyl-4-(2,2,6-trimethyl-cyclohexen-1-yl)-2-butenal, 4-(4-hydroxy-4-methylpentyl)-3-cyclohexene carbaldehyde, 4-(4-methyl-3-penten-1-yl)-3-cyclohexene carbaldehyde;

xiv) cycloaliphatic ketones, such as, for example, 1-(3,3-dimethylcyclohexyl)-4-penten-1-one, 1-(5,5-dimethyl-1-cyclohexen-1-yl)-4-penten-1-one, 2,3,8,8-tetramethyl-1,2,3,4,5,6,7,8-octahydro-2-naphtalenyl methyl-ketone, methyl-2,6,10-trimethyl-2,5,9-cyclododecatrienyl ketone, tert-butyl-(2,4-dimethyl-3-cyclohexen-1-yl)ketone;

xv) esters of cyclic alcohols, such as, for example, 2-tert-butylcyclohexyl acetate, 4-tert-butylcyclohexyl acetate, 2-tert-pentylcyclohexyl acetate, 4-tert-pentylcyclohexyl acetate, decahydro-2-naphthyl acetate, 3-pentyltetrahydro-2H-pyran-4-yl acetate, decahydro-2,5,5,8a-tetramethyl-2-naphthyl acetate, 4,7-methano-3a,4,5,6,7,7a-hexahydro-5 or 6-indenyl acetate, 4,7-methano-3a,4,5,6,7,7a-hexahydro-5 or 6-indenyl propionate, 4,7-methano-3a,4,5,6,7,7a-hexahydro-5 or 6-indenyl-isobutyrate, 4,7-methanooctahydro-5 or 6-indenyl acetate;

xvi) esters of cycloaliphatic carboxylic acids, such as, for example, allyl 3-cyclohexyl-propionate, allyl cyclohexyl oxyacetate, methyl dihydrojasmonate, methyl jasmonate, methyl 2-hexyl-3-oxocyclopentanecarboxylate, ethyl 2-ethyl-6,6-dimethyl-2-cyclohexenecarboxylate, ethyl 2,3,6,6-tetramethyl-2-cyclohexenecarboxylate, ethyl 2-methyl-1,3-dioxolane-2-acetate;

xvii) aromatic and aliphatic alcohols, such as, for example, benzyl alcohol, 1-phenylethyl alcohol, 2-phenylethyl alcohol, 3-phenylpropanol, 2-phenylpropanol, 2-phenoxyethanol, 2,2-dimethyl-3-phenylpropanol, 2,2-dimethyl-3-(3-methylphenyl)-propanol, 1,1-dimethyl-2-phenylethyl alcohol, 1,1-dimethyl-3-phenylpropanol, 1-ethyl-1-methyl-3-phenylpropanol, 2-methyl-5-phenylpentanol, 3-methyl-5-phenylpentanol, 3-phenyl-2-propen-1-ol, 4-methoxybenzyl alcohol, 1-(4-isopropylphenyl)ethanol;

xviii) esters of aliphatic alcohols and aliphatic carboxylic acids, such as, for example, benzyl acetate, benzyl propionate, benzyl isobutyrate, benzyl isovalerate, 2-phenylethyl acetate, 2-phenylethyl propionate, 2-phenylethyl isobutyrate, 2-phenylethyl isovalerate, 1-phenylethyl acetate, α-trichloromethylbenzyl acetate, α,α-dimethylphenylethyl acetate, alpha, alpha-dimethylphenylethyl butyrate, cinnamyl acetate, 2-phenoxyethyl isobutyrate, 4-methoxybenzyl acetate, araliphatic ethers, such as for example 2-phenylethyl methyl ether, 2-phenylethyl isoamyl ether, 2-phenylethyl-1-ethoxyethyl ether, phenylacetaldehyde dimethyl acetal, phenylacetaldehyde diethyl acetal, hydratropaaldehyde dimethyl acetal, phenylacetaldehyde glycerol acetal, 2,4,6-trimethyl-4-phenyl-,3-dioxane, 4,4a,5,9b-tetrahydroindeno[1,2-d]-m-dioxin, 4,4a,5,9b-tetrahydro-2,4-dimethylindeno[1,2-d]-m-dioxin;

xix) aromatic and aliphatic aldehydes, such as, for example, benzaldehyde; phenylacetaldehyde, 3-phenylpropanal, hydratropaldehyde, 4-methylbenzaldehyde, 4-methylphenylacetaldehyde, 3-(4-ethylphenyl)-2,2-dimethylpropanal, 2-methyl-3-(4-isopropylphenyl)propanal, 2-methyl-3-(4-tert-butylphenyl)propanal, 3-(4-tert-butyl-phenyl)propanal, cinnamaldehyde, alpha-butylcinnamaldehyde, alpha-amylcinnam-aldehyde, alpha-hexylcinnamaldehyde, 3-methyl-5-phenylpentanal, 4-methoxy-benzaldehyde, 4-hydroxy-3-methoxybenzaldehyde, 4-hydroxy-3-ethoxybenzaldehyde, 3,4-methylene-dioxybenzaldehyde, 3,4-dimethoxybenzaldehyde, 2-methyl-3-(4-methoxyphenyl)propanal, 2-methyl-3-(4-methylendioxyphenyl)propanal;

xx) aromatic and aliphatic ketones, such as, for example, acetophenone, 4-methylacetophenone, 4-methoxyacetophenone, 4-tert-butyl-2,6-dimethylacetophenone, 4-phenyl-2-butanone, 4-(4-hydroxyphenyl)-2-butanone, 1-(2-naphthalenyl)ethanone, benzophenone, 1,1,2,3,3,6-hexamethyl-5-indanyl methyl ketone, 6-tert-butyl-1,1-dimethyl-4-indanyl methyl ketone, 1-[2,3-dihydro-1,1,2,6-tetramethyl-3-(1-methyl-ethyl)-1H-5-indenyl]ethanone, 5′,6′,7′,8′-tetrahydro-3′,5′,5′,6′,8′,8′-hexamethyl-2-aceto-naphthone;

xxi) aromatic and araliphatic carboxylic acids and esters thereof, such as, for example, benzoic acid, phenylacetic acid, methyl benzoate, ethyl benzoate, hexyl benzoate, benzyl benzoate, methyl phenylacetate, ethyl phenylacetate, geranyl phenylacetate, phenylethyl phenylacetate, methyl cinnamate, ethyl cinnamate, benzyl cinnamate, phenylethyl cinnamate, cinnamyl cinnamate, allyl phenoxyacetate, methyl salicylate, isoamyl salicylate, hexyl salicylate, cyclohexyl salicylate, cis-3-hexenyl salicylate, benzyl salicylate, phenylethyl salicylate, methyl 2,4-dihydroxy-3,6-dimethylbenzoate, ethyl 3-phenylglycidate, ethyl 3-methyl-3-phenylglycidate;

xxii) nitrogen-containing aromatic compounds, such as, for example, 2,4,6-trinitro-1,3-dimethyl-5-tert-butylbenzene, 3,5-dinitro-2,6-dimethyl-4-tert-butylaceto-phenone, cinnamonitrile, 5-phenyl-3-methyl-2-pentenonitrile, 5-phenyl-3-methyl-pentanonitrile, methyl anthranilate, methy-N-methylanthranilate, Schiffs bases of methyl anthranilate with 7-hydroxy-3,7-dimethyloctanal, 2-methyl-3-(4-tert-butylphenyl)-propanal or 2,4-dimethyl-3-cyclohexene carbaldehyde, 6-isopropylquinoline, 6-isobutyl-quinoline, 6-sec-butylquinoline, indole, skatole, 2-methoxy-3-isopropylpyrazine, 2-iso-butyl-3-methoxypyrazine;

xxiii) phenols, phenyl ethers and phenyl esters, such as, for example, estragole, anethole, eugenol, eugenyl methyl ether, isoeugenol, isoeugenol methyl ether, thymol, carvacrol, diphenyl ether, beta-naphthyl methyl ether, beta-naphthyl ethyl ether, beta-naphthyl isobutyl ether, 1,4-dimethoxybenzene, eugenyl acetate, 2-methoxy-4-methylphenol, 2-ethoxy-5-(1-propenyl)phenol, p-cresyl phenylacetate;

xxiv) heterocyclic compounds, such as, for example, 2,5-dimethyl-4-hydroxy-2H-furan-3-one, 2-ethyl-4-hydroxy-5-methyl-2H-furan-3-one, 3-hydroxy-2-methyl-4H-pyran-4-one, 2-ethyl-3-hydroxy-4H-pyran-4-one;

xxv) lactones, such as, for example, 1,4-octanolide, 3-methyl-1,4-octanolide, 1,4-nonanolide, 1,4-decanolide, 8-decen-1,4-olide, 1,4-undecanolide, 1,4-dodecanolide, 1,5-decanolide, 1,5-dodecanolide, 1,15-pentadecanolide, cis- and trans-1-pentadecen-1,15-olide, cis- and trans-12-pentadecen-1,15-olide, 1,16-hexadecanolide, 9-hexadecen-1,16-olide, 10-oxa-1,16-hexadecanolide, 11-oxa-1,16-hexadecanolide, 12-oxa-1,16-hexadecanolide, ethylene-1,12-dodecanedioate, ethylene-1,13-tridecanedioate, coumarin, 2,3-dihydrocoumarin, and octahydrocoumarin; and

xxvi) essential oils, concretes, absolutes, resins, resinoids, balsams, tinctures such as for example ambergris tincture, amyris oil, angelica seed oil, angelica root oil, aniseed oil, valerian oil, basil oil, tree moss absolute, bay oil, armoise oil, benzoe resinoid, bergamot oil, beeswax absolute, birch tar oil, bitter almond oil, savory oil, buchu leaf oil, cabreuva oil, cade oil, calamus oil, camphor oil, cananga oil, cardamom oil, cascarilla oil, cassia oil, cassie absolute, castoreum absolute, cedar leaf oil, cedar wood oil, cistus oil, citronella oil, lemon oil, copaiba balsam, copaiba balsam oil, coriander oil, costus root oil, cumin oil, cypress oil, davana oil, dill weed oil, dill seed oil, eau de brouts absolute, oakmoss absolute, elemi oil, estragon oil, eucalyptus citriodora oil, eucalyptus oil (cineole type), fennel oil, fir needle oil, galbanum oil, galbanum resin, geranium oil, grapefruit oil, guaiacwood oil, gurjun balsam, gurjun balsam oil, helichrysum absolute, helichrysum oil, ginger oil, iris root absolute, iris root oil, jasmine absolute, calamus oil, blue camomile oil, Roman camomile oil, carrot seed oil, cascarilla oil, pine needle oil, spearmint oil, caraway oil, labdanum oil, labdanum absolute, labdanum resin, lavandin absolute, lavandin oil, lavender absolute, lavender oil, lemon-grass oil, lovage oil, lime oil distilled, lime oil expressed, linaloe oil, Litsea cubeba oil, laurel leaf oil, mace oil, marjoram oil, mandarin oil, massoi (bark) oil, mimosa absolute, ambrette seed oil, musk tincture, clary sage oil, nutmeg oil, myrrh absolute, myrrh oil, myrtle oil, clove leaf oil, clove bud oil, neroli oil, olibanum absolute, olibanum oil, opopanax oil, orange flower absolute, orange oil, origanum oil, palmarosa oil, patchouli oil, perilla oil, Peru balsam oil, parsley leaf oil, parsley seed oil, petitgrain oil, peppermint oil, pepper oil, pimento oil, pine oil, pennyroyal oil, rose absolute, rosewood oil, rose oil, rosemary oil, Dalmatian sage oil, Spanish sage oil, sandal-wood oil, celery seed oil: spike-lavender oil, star anise oil, storax oil, tagetes oil, fir needle oil, tea tree oil, turpentine oil, thyme oil, Tolu balsam, tonka bean absolute, tuberose absolute, vanilla extract, violet leaf absolute, verbena oil, vetiver oil, juniperberry oil, wine lees oil, wormwood oil, wintergreen oil, ylang-ylang oil, hyssop oil, civet absolute, cinnamon leaf oil, cinnamon bark oil, and fractions thereof or ingredients isolated therefrom.

In some embodiments, the amount of encapsulated fragrance oil is from about 80% to about 5% by weight of the total polyurea capsule suspension or capsule slurry, preferably from about 60% to about 10% by weight of the total capsule suspension or capsule slurry, and most preferably from about 50% to about 20% by weight of the total capsule suspension or capsule slurry.

In some embodiments, the amount of encapsulated fragrance oil is from about 5% to about 60% of the total weight of the hybrid encapsulate formulation, preferably from about 10% to about 50% of the total weight of the hybrid encapsulate formulation.

In addition to the fragrance materials, the present invention also contemplates the incorporation of other core additives including solvent, emollients, particles, polymeric core modifiers and/or core modifier materials encapsulated by the encapsulating polymer.

Solvents that can be used include hydrophobic materials miscible in the fragrance materials. Suitable solvents are those having reasonable affinity for the fragrance chemicals and a Clog P greater than 3.3, preferably greater than 6 and most preferably greater than 10. Suitable materials include, but are not limited to triglyceride oil, mono and diglycerides, mineral oil, silicone oil, diethyl phthalate, polyalpha olefins, castor oil and isopropyl myristate. In a highly preferred embodiment the solvent materials are combined with fragrance materials that have high Clog P values as set forth above. It should be denoted that selecting a solvent and fragrance with high affinity for each other will result in the most pronounced improvement in stability. This specific affinity may be measured by determining the Solvent-Water partition coefficient for the fragrance material. Examples include, but are not limited to, mono-, di- and tri-esters, and mixtures thereof; of fatty acids and glycerin. The fatty acid chain can range from C4-C26. Also, the fatty acid chain can have any level of unsaturation. For instance, capric/caprylic triglyceride known as NEOBEE M5 (Stepan Corporation). Other suitable examples are the CAPMUL series by Abitec Corporation, for instance CAPMUL MCM. Isopropyl myristate fatty acid esters of polyglycerol oligomers include R₂CO—[OCH₂—CH(OCOR₁)—CH₂O—]ₙ, in which R₁ and R₂ can be H or C₄-C₂₆ aliphatic chains, or mixtures thereof, and n ranges between 2-50, preferably 2-30. Nonionic fatty alcohol alkoxylates like the NEODOL surfactants by BASF, the DOBANOL surfactants by Shell Corporation or the BIOSOFT surfactants by Stepan, wherein the alkoxy group is ethoxy, propoxy, butoxy, or mixtures thereof. In addition, these surfactants can be end-capped with methyl groups in order to increase their hydrophobicity. Di- and tri-fatty acid chain containing nonionic, anionic and cationic surfactants, and mixtures thereof are also contemplated, as are fatty acid esters of polyethylene glycol, polypropylene glycol, and polybutylene glycol, or mixtures thereof. Polyalphaolefins such as the EXXONMOBIL PURESYM PAO line; esters such as the EXXONMOBIL PURESYN esters; mineral oil; silicone oils such polydimethyl siloxane and polydimethylcyclosiloxane; diethyl phthalate; and di-isodecyl adipate can also be included. In certain embodiments, ester oils have at least one ester group in the molecule. One type of common ester oil useful in the present invention are the fatty acid mono and polyesters such as cetyl octanoate, octyl isonanoanate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate; sucrose ester and polyesters, sorbitol ester, and the like. A second type of useful ester oil is predominantly composed of triglycerides and modified triglycerides. These include vegetable oils such as jojoba, soybean, canola, sunflower, safflower, rice bran, avocado, almond, olive, sesame, persic, castor, coconut, and mink oils. Synthetic triglycerides can also be employed provided they are liquid at room temperature. Modified triglycerides include materials such as ethoxylated and maleated triglyceride derivatives provided they are liquids. Proprietary ester blends such as those sold by FINETEX as FINSOLV are also suitable, as is ethylhexanoic acid glyceride. A third type of ester oil is liquid polyester formed from the reaction of a dicarboxylic acid and a diol. Examples of polyesters suitable for the present invention are the polyesters marketed by EXXONMOBIL under the trade name PURESYN ESTER.

Nanoscale solid particulate materials such as those disclosed in U.S. Pat. No. 7,833,960 may also be incorporated into the core and may be selected from, but not limited to, metal or metallic particles, metal alloys, polymer particles, wax particles, inorganic particulates, minerals and clay particles.

The metal particles can be selected from a non-limiting list of main group elements, transition metal and post-transition metal elements including aluminum (Al), silica (Si), Titanium (Ti), chromium (Cr), manganese (Mn), iron (Fe), nickel (Ni), cobalt (Co), copper (Cu), gold (Au), silver (Ag), platinum (Pt) and palladium (Pd).

Polymer particles of any chemical composition and nature are suitable for the present invention as long as their physical dimension falls into the prescribed region and a liquid core is generated. The polymer particles can be selected from a nonlimiting list of polymers and co-copolymer based on polystyrene, polyvinyl acetate, polylactides, polyglycolides, ethylene maleic anhydride copolymer, polyethylene, polypropylene, polyamide, polyimide, polycarbonate, polyester, polyurethane, polyurea, cellulose and cellulose, and combinations and mixture of such polymers.

The inorganic particulate can be selected from a non-limiting list including silica, titanium dioxide (TiO₂), zinc oxide (ZnO), Fe₂O₃, and other metal oxides (e.g., NiO, Al₂O₃, SnO, SnO₂, CeO₂, ZnO, CdO, RuO₂, FeO, CuO, AgO, MnO₂, and other transition metal oxides).

Examples of nanoscaled material include AEROSIL R812, which has a particle size of less than 25 nm according to the specification from the manufacture, Degussa Corp. Other suitable materials from Degussa include, but not limited to, AEROSIL R972, AEROSIL R974, AEROSIL R104, AEROSIL R106, AEROSIL R202, AEROSIL R805, AEROSIL R812, AEROSIL R812S, AEROSIL R816, AEROSIL R7200, AEROSIL R9200, and AEROXIDE TiO₂ P25, AEROXIDE T805, AEROXIDE LE1, AEROXIDE LE2, AEROXIDE TiO₂ NKT 90, AEROXIDE Alu C805, titanium dioxide PF2, SIPERNAT D110, SIPERNAT D-380. The hydrophobic materials from Deguassa Corp. such as including AEROSILE R812 and R972 are especially preferred.

Nanoscaled materials such as UVINUL TiO₂ and Z-COTE HP1 manufactured by BASF can also be used as well as and TI-PURE titanium dioxide, TI-PURE R-700, and TI-SELECT. Additional suitable materials include TS-6200 from Dupont and ZEROFREE 516, HUBERDERM 2000 and HUBERDERM 1000 from the J. M. Huber Corporation, Havre De Grace, MD. Silica products such as SYLOID 63, 244, 72, 63FP, 244FP, 72FP, SYLOX 15, 2 and Zeolites such as SYLOSIV A3, SYLOSIV A4 and SYLOSIV K300 from Grace Davison can also be used.

Polymeric core modifiers are also contemplated. It has been found that the addition of hydrophobic polymers to the core can also improve stability by slowing diffusion of the fragrance from the core. The level of polymer is normally less than 80% of the core by weight, preferably less than 50%, and most preferably less than 20%. The basic requirement for the polymer is that it be miscible or compatible with the other components of the core, namely the fragrance and other solvent. Preferably, the polymer also thickens or gels the core, thus further reducing diffusion. Polymeric core modifiers include copolymers of ethylene; copolymers of ethylene and vinyl acetate (e.g., ELVAX polymers by DOW Corporation); copolymers of ethylene and vinyl alcohol (EVAL polymers by Kuraray); ethylene/acrylic elastomers (e.g., VALNAC polymers by Dupont); polyvinyl polymers (e.g., polyvinyl acetate); alkyl-substituted cellulose (e.g., ethyl cellulose such as ETHOCEL made by DOW Corporation, and hydroxypropyl celluloses such as KLUCEL polymers made by Hercules); cellulose acetate butyrate available from Eastman Chemical; polyacrylates (e.g., AMPHOMER, DEMACRYL LT and DERMACRYL 79, made by National Starch and Chemical Company, the AMERHOLD polymers by Amerchol Corporation, and ACUDYNE 258 by ISP Corporation); copolymers of acrylic or methacrylic acid and fatty esters of acrylic or methacrylic acid (e.g., INTELIMER POLYMERS made by Landec Corporation; see also U.S. Pat. Nos. 4,830,855, 5,665,822, 5,783,302, 6,255,367 and 6,492,462); polypropylene oxide; polybutylene oxide of poly(tetrahydrofuran); polyethylene terephthalate; polyurethanes (e.g., DYNAM X by National Starch); alkyl esters of poly(methyl vinyl ether); maleic anhydride copolymers (e.g., the GANTREZ copolymers and OMNIREZ 2000 by ISP Corporation; carboxylic acid esters of polyamines e.g., ester-terminated polyamides (ETPA) made by Arizona Chemical Company); polyvinyl pyrrolidone (LUVISKOL series of BASF); block copolymers of ethylene oxide, propylene oxide and/or butylenes oxide including, e.g., PLURONIC and SYNPERONIC polymers/dispersants by BASF. Another class of polymers include polyethylene oxide-co-propyleneoxide-co-butylene oxide polymers of any ethylene oxide/propylene oxide/butylene oxide ratio with cationic groups resulting in a net theoretical positive charge or equal to zero (amphoteric). The general structure is:

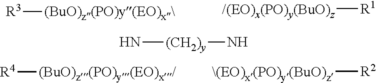

where R¹, R², R³, and R⁴ are independently H or any alkyl or fatty alkyl chain group. Examples of such polymers are the commercially known as TETRONICS by BASF Corporation.

Sacrificial core ingredients can also be included. These ingredients are designed to be lost during or after manufacture and include, but are not limited to, highly water soluble or volatile materials.

The level of solvent materials, particles or polymeric core modifiers in the core encapsulated by the encapsulating polymer should be greater than about 10 weight percent, preferably greater than about 30 weight percent and most preferably greater than about 70 weight percent. In addition to the solvent, it is preferred that higher Clog P fragrance materials are employed. It is preferred that greater than about 60 weight percent, preferably greater than 80 and more preferably greater than about 90 weight percent of the fragrance chemicals have Clog P values of greater than about 3.3, preferably greater than about 4 and most preferably greater than about 4.5. Those with skill in the art will appreciate that many formulations can be created employing various solvents and fragrance chemicals. The use of a high level of high Clog P fragrance chemicals will likely require a lower level of hydrophobic solvent than fragrance chemicals with lower Clog P to achieve similar performance stability. As those with skill in the art will appreciate, in a highly preferred embodiment, high Clog P fragrance chemicals and hydrophobic solvents comprise greater than about 80, preferably more than about 90 and most preferably greater than 95 weight percent of the fragrance composition. As discussed above, specific Clog P values may be measured between candidate solvents and water for the fragrance materials to be included in the core. In this way, an optimum solvent choice may be made. In fact, since most fragrances will have many ingredients, it may be preferable to measure the partitioning of a specific fragrance blend in solvent and water in order to determine the effect of any material interactions.

Other active materials that can be included the in capsules of this invention include antimicrobial agents such as thymol, 2-hydroxy-4,2,4-trichlorodiphenylether, triclocarban; organic sunscreen actives such as oxybenzone, octylmethoxy cinnamate, butylmethoxy dibenzoyln ethane, p-aminobenzoic acid and octyl dimethyl-p-aminobenzoic acid; vitamins such as Vitamin A, Vitamin C and Vitamin E or esters thereof; and malodor counteracting ingredients including, but not limited to, an α,β-unsaturated carbonyl compounds including but not limited to those disclosed in U.S. Pat. No. 6,610,648 and EP 2,524,704, amyl cinnamaldehyde, benzophenone, benzyl benzoate, benzyl isoeugenol, benzyl phenyl acetate, benzyl salicylate, butyl cinnamate, cinnamyl butyrate, cinnamyl isovalerate, cinnamyl propionate, decyl acetate, ethyl myristate, isobutyl cinnamate, isoamyl salicylate, phenethyl benzoate, phenethyl phenyl acetate, triethyl citrate, tripropylene glycol n-butyl ether, isomers of bicyclo[2.2.1]hept-5-ene-2-carboxylic acid, ethyl ester, and zinc undecenylate.

As used herein olfactory effective amount is understood to mean the amount of compound in perfume compositions the individual component will contribute to its particular olfactory characteristics, but the olfactory effect of the fragrance composition will be the sum of the effects of each of the fragrance ingredients. Thus, the fragrances of the invention can be used to alter the aroma characteristics of the perfume composition by modifying the olfactory reaction contributed by another ingredient in the composition. The amount will vary depending on many factors including other ingredients, their relative amounts and the effect that is desired.

Spray-drying typically includes breaking up an emulsion into droplets of desired size, e.g., in a spray nozzle, from a spinning disc, or apertured centrifugal atomizer, and removing moisture in a drying environment to solidify the coating material in the droplets to form solid particles. The drying environment preferably is hot drying air, e.g., in a spray-drying tower. The particles produced by this process, are characterized by a cellular structure composed of many dispersed globules of the core material in a matrix of the coating material. Any suitable method of spray-drying can be used in conjunction with this invention including, but is not limited to, spray-drying tower or continuous fluidized bed spray granulation (see, for example, WO 00/36931). Useful spray towers include dryers from Anhydro, Niro or Nubilosa.

Applications. The present invention is well-suited for use in personal care products including, without limitation, deodorants and antiperspirants, shampoos, hair conditioners, hair rinses, hair refreshers, body washes, soaps products and the like. In particular embodiments, the formulation of the invention is of use in an aerosol antiperspirant, stick antiperspirant, roll-on antiperspirant, emulsion spray antiperspirant, clear emulsion stick antiperspirant, soft solid antiperspirant, emulsion roll-on antiperspirant, clear emulsion stick antiperspirant, opaque emulsion stick antiperspirant, clear gel antiperspirant, clear stick deodorant or spray deodorant. Exemplary personal care product formulations are provided in Examples 3-9.

The present invention is also well-suited for use in fabric care products such as rinse conditioners and liquid and powder detergents; home care products such as all-purpose cleaners and fabric refreshers; personal hygiene products such as hand sanitizers; toiletries; and oral care products such as tooth powder, all of which are known in the art. For example, liquid laundry detergents include those systems described in U.S. Pat. Nos. 5,929,022, 5,916,862, 5,731,278, 5,565,145, 5,470,507, 5,466,802, 5,460,752, 5,458,810, 5,458,809, 5,288,431, 5,194,639, 4,968,451, 4,597,898, 4,561,998, 4,550,862, 4,537,707, 4,537,706, 4,515,705, 4,446,042, and 4,318,818. Liquid dish detergents are described in U.S. Pat. Nos. 6,069,122 and 5,990,065.

The invention is described in greater detail by the following non-limiting examples.

### Example 1

### Hybrid Spray Fragrance Encapsulation Formulations

Hybrid polyurea capsule/starch formulations were prepared as follows.

Preparation of Part A. Part A was prepared by weighing out the desired amount of tap water and CAPSUL Starch (National Starch, Bridgewater, N.J.) into a suitable container. The mixture was then heated to 50-55° C. and Maltose (Mitsubishi, Japan) and METHOCEL cellulose ethers (Dow Chemical, Middle Land, Mich.) were added. The mixture was kept at 55° C. and stirred with an overhead mixer until a homogeneous solution was obtained. Part A was cooled to 19° C. by submersion in an ice bath to prevent pre-mature volatilization of fragrance ingredients.

Preparation of Part B. Part B was prepared by weighing out the desired amount of DIMODAN PH320 (distilled monoglyceride; Dow Chemical, Middle Land, Mich.) and heating until the material was liquefied. The desired amount of fragrance was then added with constant mixing until a homogenous phase was obtained.

Preparation of Part C. Part C was prepared by preparing a 30% LUVISKOL K-30 (Polyvinylpyrrolidone; BASF) by dissolving solid LUVISKOL K30 into deionized water. The solution was then mixed into polyurea capsule slurry, which had been pH-adjusted to between 7 and 8, under constant stirring. The mixing continued for an additional 30 minutes to ensure a homogenous mixture was obtained.

Preparation of Fragrance Emulsion. A fragrance emulsion was prepared by adding Part B or fragrance oil into Part A. The mixture was subjected to mixing with a high shear homogenizer (Greerco, Model 11, 2001 with Baldor Industrial Motor), while still submerged in an ice-bath. The prepared emulsion had a particle size of 3 microns or less.

Preparation of Solution for Spray Drying. The mixture of Part C was combined with the fragrance emulsion under consistent stirring with an overhead mixer. This mixture was then fed into a Niro Spray Drier. The inlet temperature was maintained at 190° C. and the emulsion was fed at a rate sufficient to maintain an exit air temperature at 90° C.

Formulations containing different ratios of fragrance emulsion to capsule slurry were prepared, each containing a 45.1% fragrance load. Formula 1 (Table 1; 90/10 ratio) and Formula 2 (Table 2; 70/30 ratio) were prepared as above. Formula 3 (Table 3; 90/10 ratio) and Formula 4 (Table 4; 70/30 ratio) were prepared as above, however, the fragrance was added directly to the starch solution. Formula 5 (Table 5; 90/10 ratio) and Formula 6 (Table 6; 70/30 ratio) were prepared as above, however, the fragrance was added directly to the starch solution, which was composed of HI-CAP 100 (modified food starch derived from waxy maize).

In the context of this invention, the ratio of starch/core-shell capsule is defined as the dry weight of starch to that of the core-shell capsule suspension minus the amount of water in the suspension. Thus, the dry weight of the core-shell capsule is the cumulative weight of the capsule wall polymer, the oil/fragrance core, and the capsule formation aid used in preparing the core-shell capsule.

TABLE 1

| Part | Ingredient | Parts* | Dry Weight (%) |
|---|---|---|---|
| A | City Water | 880.00 | |
| | CAPSUL Starch | 416.00 | 41.60 |
| | Maltose | 41.20 | 4.12 |
| | METHOCEL A4M | 23.00 | 2.30 |
| B | DIMODAN PH 320 | 17.00 | 1.70 |
| | Fragrance | 382.80 | 38.28 |
| C | LUVISKOL K-30 30% soln | 20.00 | 2.00 |
| | Polyurea capsule slurry | 100.00 | 10.00 |
| | Total Solid Input: | 1000.00 | 100.00 |

*Unit: grams

TABLE 2

| Part | Ingredient | Parts* | Dry Weight (%) |
|---|---|---|---|
| A | City Water | 665.00 | |
| | CAPSUL Starch | 383.50 | 38.35 |
| | Maltose | 46.00 | 4.60 |
| | METHOCEL A4M | 23.00 | 2.30 |
| B | DIMODAN PH 320 | 13.00 | 1.30 |
| | Fragrance | 199.50 | 19.95 |
| C | LUVISKOL K-30 30% soln | 35.00 | 3.50 |
| | Polyurea capsule slurry | 300.00 | 30.00 |
| | Total Solid Inputs: | 1000.00 | 100.00 |

*Unit: grams

TABLE 3

| Part | Ingredient | Parts* | Dry Weight (%) |
|---|---|---|---|
| A | City Water | 834.00 | |
| | Capsule Starch | 452.00 | 45.20 |
| | Maltose | 46.00 | 4.60 |
| | Fragrance | 382.00 | 38.20 |
| B | LUVISKOL K-3G 30% soln | 20.00 | 2.00 |
| | Polyurea capsule slurry | 100.00 | 10.00 |
| | Total Solid Inputs: | 1000.00 | 100.00 |

*Unit: grams

TABLE 4

| Part | Ingredient | Parts* | Dry Weight (%) |
|---|---|---|---|
| A | City Water | 665.00 | |
| | Capsule Starch | 411.50 | 41.15 |
| | Maltose | 54.00 | 5.40 |
| | Fragrance | 199.50 | 19.95 |
| B | LUVISKOL K-30 30% soln | 35.00 | 3.50 |
| | Polyurea capsule slurry | 300.00 | 30.00 |
| | Total Solid Inputs: | 1000.00 | 100.00 |

*Unit: grams

TABLE 5

| Part | Ingredient | Parts* | Dry Weight (%) |
|---|---|---|---|
| A | City Water | 880.00 | |
| | HI-CAP 100 | 498.00 | 49.80 |
| | Fragrance | 382.00 | 38.20 |
| B | LUVISKOL K-30 30% soln | 20.00 | 2.00 |
| | Polyurea capsule slurry | 100.00 | 10.00 |
| | Total Solid Inputs: | 1000.00 | 100.00 |

*Unit: grams

TABLE 6

| Part | Ingredient | Parts* | Dry Weight (%) |
|---|---|---|---|
| A | City Water | 665.00 | |
| | HI-CAP 100 | 415.00 | 41.50 |
| | Fragrance | 250.00 | 25.00 |
| B | LUVISKOL K-30 30% soln | 35.00 | 3.50 |
| | Polyurea capsule slurry | 300.00 | 30.00 |
| | Total Solid Inputs: | 1000.00 | 100.00 |

*Unit: grams

### Example 2

### Sensory Performance of Hybrid Spray Fragrance Encapsulation Formulations

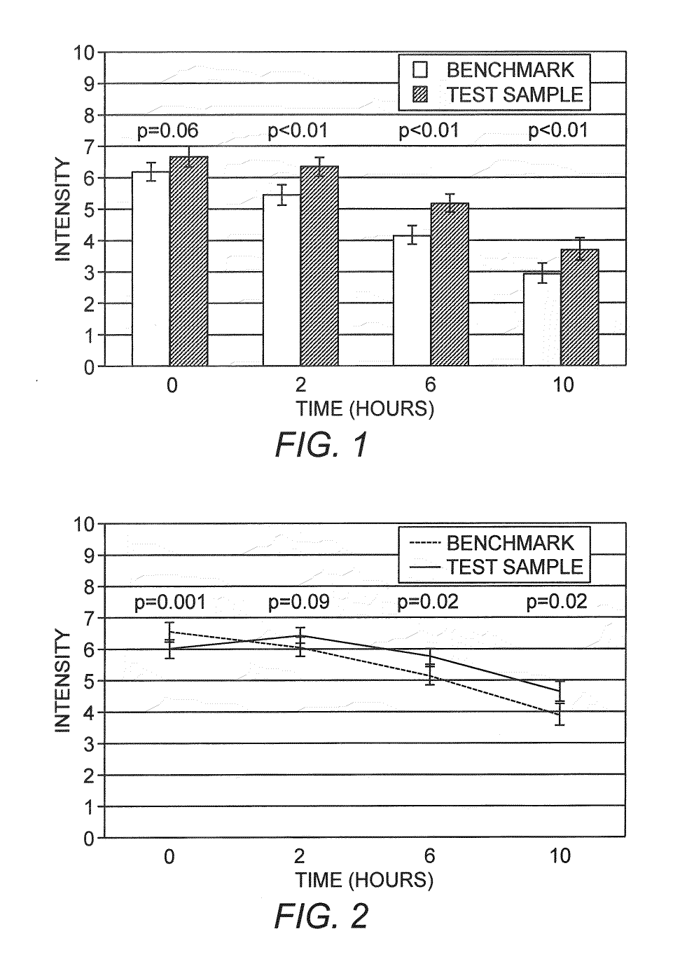

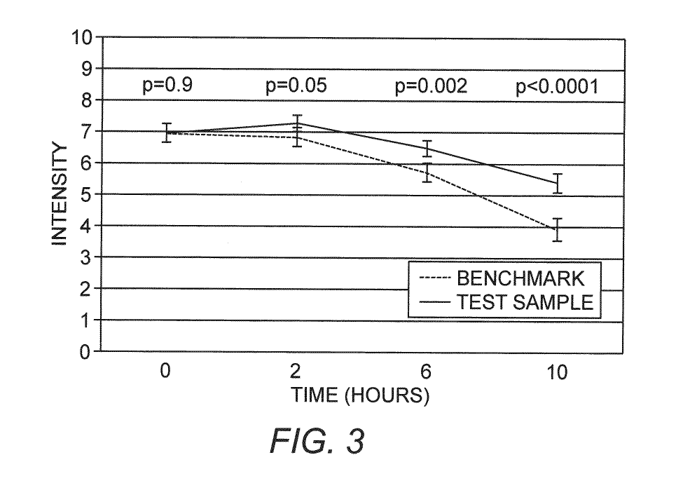

Hybrid polyurea capsule formulations were prepared in various bases and performance was evaluated against benchmark samples (market samples). Performance was evaluated by an internal sensory protocol as follows. Panelists (30-35, with a mix of male and female) were instructed to shower with an unfragranced soap on the day of evaluation. For the comparative analysis, one underarm was applied with the test sample, the other with a benchmark sample. The test samples included a hybrid polyurea capsule formulation composed of 46% ICON fragrance at 0.1% (fragrance equivalent load) in an antiperspirant aerosol base (FIG. 1), a hybrid polyurea capsule formulation composed of TORNADO fragrance at 0.25% NOE in antiperspirant aerosol base (FIG. 2), and a hybrid polyurea capsule formulation composed of No LIMIT fragrance at 0.25% NOE in antiperspirant aerosol base (FIG. 3), each of which was weighed and wrapped in wax paper for easy application onto skin. Application of the samples was counterbalanced across underarms. Fragrance intensity was evaluated at 0, 2, 6 and 10 hours after application on a 0-10 intensity scale. Intensity ratings were entered by panelists into an automated data entry system, (COMPUSENSE at-hand) at the designated times. Intensity scores were averaged across panelists for each sample and analyzed by Two-Way ANOVA (p<0.1/90% CI). The results of this analysis are presented in FIGS. 1-3 and show that the hybrid polyurea capsule formulation showed significantly better performance.

### Example 3

### Preparation of Polyurea Capsules Using Different Dispersants

Preparation of Aqueous Phase. Fifty grams of 6% wt desired emulsifiers and dispersants were added into 169.2 g DI water to form an aqueous phase.

Emulsion of Aqueous Phase and Fragrance Oil Phase. Isocyanate M20 (19.2 g) was dissolved in a mixture of 192 g Woody and 48 g NEOBEE to form a fragrance oil phase. The aqueous phase and fragrance oil phase were homogenized at 9500 rpm for 3 minutes to form an emulsion.

Formation of Fragrance Capsules. The emulsion was placed in 1000 ml round bottom vessel and 21.6 g of 40% HMDA was added under constant mixing with an overhead mixer as the emulsion was heated to 35° C. The resulting capsule slurry was heated to 55° C. and cured at 55° C. for two hours. The results of the preparations are provided in Table 7.

TABLE 7

| Formulation | Capsule Aid | Chemical Nature | Results |
|---|---|---|---|
| 1 | PAA | Anionic, polyacrylate | Fail |
| 2 | Alginate | Anionic polymer | Fail |
| 3 | LAS | Anionic surfactant, linear | Fail |
| | | alkylbenzene sulfonate | |
| 4 | PSSS | Anionic, polystyrene sulfonic | Free |
| | | acid, sodium salt | oil <1.0% |
| 5 | ZEMAC | Anionic, poly(ethylene-co- | Free |
| | | maleic anhydride) | oil <1.10 |

PAA, polyacrylic acid; LAS, linear alkylbenzene sulfonate; PSSS, sulfonated polystyrene.

### Example 4

### Clear Deodorant Stick Formulation

TABLE 8

| Ingredient | Percentage |
|---|---|
| Water | 20 |
| Phosphatidylglycerol/Diphosphatidylglycerol | 55 |
| Sodium Stearate | 6 |
| PEG-4 | 15 |
| Antibacterial Agent | 0.1 |

### Example 5

### Antiperspirant Emulsion Spray Formulation

An exemplary antiperspirant emulsion spray formulation is provided in Table 9.

TABLE 9

| Ingredient | Percentage |
|---|---|
| Water | to 100 |
| Dimethicone | 6 |
| Aluminum Chlorohydrate | 5-6 |
| EDTA | 0.15 |
| Lauryl PEG-9 Polydimethylsiloxyethyl Dimethicone | 0.3 |
| Phenoxyethanol | |
| Isobutane | 0.3 |
| | 70 |

### Example 6

### Antiperspirant Emulsion Roll-On Formulation

An exemplary antiperspirant emulsion roll-on formulation is provided in Table 10.

TABLE 10

| Ingredient | Percentage |
|---|---|
| Water | to 100 |
| Aluminum Chlorohydrate or Aluminum Zirconium | 32-36 |
| Tetrachlorohydrex Gly | |
| Steareth-2, Steareth-20 | 0.5-4 |
| Silica | 1-5 |
| Glycerin | 3-5 |
| Dimethicone | 0.5 |

### Example 7

### Antiperspirant Clear Emulsion Stick Formulation

An exemplary antiperspirant clear emulsion stick formulation is provided in Table 11.

TABLE 11

| Ingredient | Percentage |
|---|---|
| Water | 40 |
| Aluminum Zirconium Tetrachlorohydrex Gly | 20 |
| Stearyl Alcohol | 30 |
| C12-C15 Alkyl Benzoate | 25 |
| Glycine | 7 |
| Dimethicone | 0.07 |

### Example 8

### Antiperspirant Opaque Emulsion Stick Formulation

An exemplary antiperspirant opaque emulsion stick formulation is provided in Table 12.

TABLE 12

| Ingredient | Percentage |
|---|---|
| Water | to 100 |
| Aluminum Chlorohydrate | 40 |
| Isopropyl Palmitate | 9 |
| Dimethicone | 5.8 |
| Synthetic Wax | 9 |
| Beheneth-10 | 2 |
| Polyglyceryl-3 Diisosterate | 0.3 |
| Acrylates Copolymer | 0.3 |
| PEG/PPG-18/18 Dimethicone | 2 |
| Phenoxyethanol | 0.5 |
| Pentylene Glycol | 0.5 |
| Cetyl PEG/PPG-10/1 Dimethicone | 2 |

### Example 9

### Deodorant Spray Formulation

An exemplary deodorant spray formulation is provided in Table 13.

TABLE 13

| Ingredient | Percentage |
|---|---|
| Denatured Alcohol | 45 |
| Polyaminopropyl biguanide stearate | 0.2-0.5 |
| Butane, Isobutane, Propane, 152A | 55 |

### Example 10

### Antiperspirant Clear Gel Formulation

An exemplary antiperspirant clear gel formulation is provided in Table 14.

TABLE 14

| Ingredient | Percentage |
|---|---|
| Water | 20 |
| Aluminum Zirconium Tetrachlorohydrex Gly | 25 |
| Silicone | 40 |
| Phosphatidylglycerol | 10 |
| Emulsifier | 10 |

What is claimed is:

1. A hybrid encapsulate formulation obtained by a method comprising
(a) preparing an aqueous starch solution;
(b) preparing an oil phase containing an active material;
(c) emulsifying the oil phase with the aqueous starch solution to obtain an emulsion;
(d) mixing the emulsion with a polyurea capsule suspension; and
(e) spray drying the mixture to obtain a hybrid encapsulate formulation.

2. The hybrid encapsulate formulation of claim 1, wherein the aqueous starch solution further contains maltose, sucrose, maltodextrin, or a combination thereof.

3. The hybrid encapsulate formulation of claim 1, wherein the oil phase further contains monoglycerides, lecithin, or a combination thereof.

4. The hybrid encapsulate formulation of claim 1, wherein the active material is a fragrance oil.

5. The hybrid encapsulate formulation of claim 1, wherein the polyurea capsule encapsulates an active material.

6. The hybrid encapsulate formulation of claim 1, wherein the polyurea capsule suspension is washed with water prior to being mixed with the emulsion.

7. The hybrid encapsulate formulation of claim 6, wherein a salt is added to the polyurea capsule suspension before washing the polyurea capsule suspension with water.

8. The hybrid encapsulate formulation of claim 5, wherein the active material is a fragrance oil.

9. The hybrid encapsulate formulation of claim 5, wherein the active material in the emulsion and the active material in the polyurea capsule are the same.

10. The hybrid encapsulate formulation of claim 5, wherein the active material in the emulsion and the active material in the polyurea capsule are different.

11. The hybrid encapsulate formulation of claim 1, wherein the polyurea capsule suspension comprises a nonionic polymer, a cationic polymer, an anionic polymer, anionic surfactant, or a combination thereof.

12. The hybrid encapsulate formulation of claim 11, wherein the nonionic polymer is polyvinylpyrrolidone, polyvinyl alcohol, polyethylene glycol, polyethylene oxide, polyethylene oxide-polypropylene oxide, polyethylene oxide-polypropylene oxide-polyethylene oxide, or a combination thereof.

13. The hybrid encapsulate formulation of claim 1, wherein the cationic polymer is Polyquaternium-11, Polyquaternium-6, Polyquaternium-47, or a combination thereof.

14. The hybrid encapsulate formulation of claim 11, wherein the anionic polymer is polystyrene sulfonic acid, polyacrylic acid, hyaluronic acid, sodium alginate, sodium carboxymethylcellulose, or a combination thereof.

15. The hybrid encapsulate formulation of claim 11, wherein the anionic surfactant is sodium laureth sulfate, complex ester of phosphoric acid and ethoxylated cosmetic grade oleyl alcohol, or a combination thereof.

16. The hybrid encapsulate formulation of claim 1, wherein the starch/polyurea capsule are at a ratio in the range of 10/90 to 90/10.

17. A personal care product comprising the hybrid encapsulate formulation of claim 1.

18. The personal care product of claim 17, wherein said product is an aerosol antiperspirant, stick antiperspirant, roll-on antiperspirant, emulsion spray antiperspirant, clear emulsion stick antiperspirant, soft solid antiperspirant, emulsion roll-on antiperspirant, clear emulsion stick antiperspirant, opaque emulsion stick antiperspirant, clear gel antiperspirant, clear stick deodorant or spray deodorant.

19. The personal care product of claim 17, wherein said product is a shampoo, hair conditioner, hair rinse, hair refresher, body wash or soap.

20. A beauty care product comprising the hybrid encapsulate formulation of claim 1.

21. The beauty care product of claim 20, wherein said product is a fine fragrance or Eau De Toilette product.

22. A fabric care product comprising the hybrid encapsulate formulation of claim 1.

23. The fabric care product of claim 22, wherein said product is a rinse conditioner, liquid detergent or powder detergent.

24. A home care product comprising the hybrid encapsulate formulation of claim 1.

25. The home care product of claim 24, wherein said product is an all-purpose cleaner or fabric refresher.

26. A personal hygiene product comprising the hybrid encapsulate formulation of claim 1.

27. The personal hygiene product of claim 26, wherein the product is a hand sanitizer.

28. An oral care product comprising the hybrid encapsulate formulation of claim 1.

29. The oral care product of claim 28, wherein the product is a tooth powder.

30. A method for releasing an encapsulated fragrance by moisture, shear, or a combination thereof comprising
(a) encapsulating a first fragrance in a polyurea capsule,
(b) mixing the polyurea encapsulated fragrance with a fragrance emulsion comprising a second fragrance and starch to obtain a hybrid encapsulate formulation,
(c) spray drying the hybrid encapsulate formulation,
(d) incorporating the dried hybrid fragrance encapsulate formulation into a consumer product base to form a consumer product,
(e) applying the consumer product to a surface, and
(f) exposing the surface to moisture, shear, or a combination thereof so that the encapsulated fragrance is released.

31. The method of claim 30, wherein the fragrance emulsion further comprises maltose, sucrose, maltodextrin, or a combination thereof.

32. The method of claim 30, wherein the fragrance emulsion further comprises monoglycerides, lecithin, or a combination thereof.

33. The method of claim 30, wherein the polyurea capsule is in a suspension comprising a nonionic polymer, a cationic polymer, an anionic polymer, anionic surfactant, or a combination thereof.

34. The method of claim 33, wherein the nonionic polymer is polyvinylpyrrolidone, polyvinyl alcohol, polyethylene glycol, polyethylene oxide, polyethylene oxide-polypropylene oxide, polyethylene oxide-polypropylene oxide-polyethylene oxide, or a combination thereof.

35. The method of claim 33, wherein the cationic polymer is Polyquaternium-11, Polyquaternium-6, Polyquaternium-47, or a combination thereof.

36. The method of claim 33, wherein the anionic polymer is polystyrene sulfonic acid, polyacrylic acid, hyaluronic acid, sodium alginate, sodium carboxymethylcellulose, or a combination thereof.

37. The method of claim 33, wherein the anionic surfactant is sodium laureth sulfate, complex ester of phosphoric acid and ethoxylated cosmetic grade oleyl alcohol, or a combination thereof.

38. The method of claim 30, wherein the first fragrance and second fragrance are the same.

39. The method of claim 30, wherein the first fragrance and second fragrance are the different.

40. The method of claim 30, wherein the starch/polyurea capsule are at a ratio in the range of 10/90 to 90/10.

41. A method for preparing a hybrid encapsulate formulation comprising
(a) preparing an aqueous starch solution;
(b) preparing an oil phase containing an active material;
(c) emulsifying the oil phase with the aqueous starch solution to obtain an emulsion;
(d) mixing the emulsion with a polyurea capsule suspension; and
(e) spray drying the mixture to obtain a hybrid encapsulate formulation.

42. The method of claim 41, wherein the aqueous starch solution further comprises maltose, sucrose, maltodextrin, or a combination thereof.

43. The method of claim 41, wherein the oil phase further comprises monoglycerides, lecithin, or a combination thereof.

44. The method of claim 41, wherein the active material is a fragrance oil.

45. The method of claim 41, further comprising the step of washing the polyurea capsule suspension with water prior to mixing the emulsion with the polyurea capsule suspension.

46. The method of claim 45, further comprising the step of adding a salt to the polyurea capsule suspension before washing the polyurea capsule suspension with water.

47. The method of claim 41, wherein the polyurea capsule encapsulates an active material.

48. The method of claim 47, wherein the active material is a fragrance oil.

49. The method of claim 47, wherein the active material in the emulsion and the active material in the polyurea capsule are the same.

50. The method of claim 47, wherein the active material in the emulsion and the active material in the polyurea capsule are different.

51. The method of claim 41, wherein the polyurea capsule suspension comprises a nonionic polymer, a cationic polymer, an anionic polymer, anionic surfactant, or a combination thereof.

52. The method of claim 51, wherein the nonionic polymer is polyvinylpyrrolidone, polyvinyl alcohol, polyethylene glycol, polyethylene oxide, polyethylene oxide-polypropylene oxide, polyethylene oxide-polypropylene oxide-polyethylene oxide, or a combination thereof.

53. The method of claim 51, wherein the cationic polymer is Polyquaternium-11, Polyquaternium-6, Polyquaternium-47, or a combination thereof.

54. The method of claim 51, wherein the anionic polymer is polystyrene sulfonic acid, polyacrylic acid, hyaluronic acid, sodium alginate, sodium carboxymethylcellulose, or a combination thereof.

55. The method of claim 51, wherein the anionic surfactant is sodium laureth sulfate, complex ester of phosphoric acid and ethoxylated cosmetic grade oleyl alcohol, or a combination thereof.

56. The method of claim 41, wherein the starch/polyurea capsule are at a ratio in the range of 10/90 to 90/10.
